(19) United States
(12) Patent Application Publication
(10) Pub. No.: US 2022/0387806 A1
(43) Pub. Date: Dec. 8, 2022

(54) CONDUCTIVE WIRELESS POWER SYSTEMS

(71) Applicant: Auckland Uniservices Limited, Auckland (NZ)

(72) Inventors: John Daniel McCormick, Waiheke Island (NZ); David Mortimer Budgett, Auckland (NZ); Reza Sedehi, Auckland (NZ); Aiguo Hu, Auckland (NZ)

(21) Appl. No.: 17/845,025

(22) Filed: Jun. 21, 2022

Related U.S. Application Data
(63) Continuation of application No. PCT/IB2020/062264, filed on Dec. 21, 2020.

(30) Foreign Application Priority Data
Dec. 20, 2019 (NZ) 760495

(51) Int. Cl. A61N 1/378; A61N 1/372; A61N 1/05; H02J 50/10; H02J 50/00; H02J 50/80; H02J 50/70; H04B 13/00

(52) U.S. Cl. CPC A61N 1/3787; A61N 1/37205; A61N 1/057; H02J 50/10; H02J 50/005; H02J 50/80; H02J 50/70; H04B 13/005

(57) ABSTRACT

A wireless power transfer system that employs a form of conductively coupled power transfer to transfer energy to deeply implanted devices.

### FIELD

This disclosure relates to wireless power transfer using conductive or conductive/capacitive techniques or systems. It also relates to implantable medical devices, and in particular to energy transfer to these devices or implants by transferring power through human or animal tissues.

### BACKGROUND

Implantable medical devices (IMDs), also known as biomedical implants are in common use in medicine for diagnostic and therapeutic purposes. An active device or implant is a medical device that is equipped for its functioning with a source of electrical energy and is totally or partially introduced, surgically or medically into the human (or other animal) body. Thus active biomedical implants require power to operate. The power requirement of such devices, ranging from microwatts to 10's several of Watts, can either be supplied by implanted energy storage units or via percutaneous (through the skin) drive lines. Due to the short lifetime, the size of batteries or similar storage units or, and infection potential of percutaneous drive lines, wireless power transfer (WPT) has become the preferred long term power source for implantable devices.

At present, the most commonly used type of transcutaneous WPT is inductive power transfer (IPT). Inductive power transfer uses a magnetic field to couple an external and implanted coil to deliver power. However, the implanted coils are difficult to integrate into miniaturized implants due to the presence of metallic components such as the battery or hermetic packaging, which creates parasitic loads that reduce performance. Moreover, achieving high power densities that assist with miniaturization leads to high field strengths and circulating currents, which makes it difficult to meet specific absorption rate limits (SAR).

Capacitive power transfer systems have been developed for wireless/contactless power transfer. They use adjacent pairs of external surface electrodes to generate an electromotive force, with corresponding adjacent pick-up electrodes just below the skin surface. Whilst this method delivers useful amounts of power to implants which are used near the body surface, the voltage potential applied to deliver power, leads to large highly undesirable surface currents if the implant receiving the available power is implanted deeply.

Object

It is an object of the present invention to provide an improved wireless power transfer system or method, or to provide improved implantable devices.

### SUMMARY

In one aspect the disclosed subject matter provides an implantable device implantable via a vessel in the body, the implantable device comprising external conductive features and being configured to receive power via the conductive features from an electromotive force applied to body tissue.

Preferably an AC waveform is used to apply the EMF to the tissue for power transfer.

Preferably the conductive features comprise one or more exterior conductive surfaces.

Preferably the conductive features are separated by an insulator

Preferably one or more of the conductive features may comprise a locating feature of the device.

Preferably one or more of the conductive features may comprise an anchoring feature of the device.

Preferably one of more of the conductive features may perform a conductive function in addition to receiving power.

Preferably one of more of the conductive features may additionally comprise a pacemaker electrode.

Preferably one of more of the conductive features may additionally comprise a neurostimulator.

Preferably the device receives power using a device fixation feature which anchors the device to a vessel or organ wall.

Preferably the device receives power using a delivery feature as one of the electrodes, the delivery feature being used to temporarily attach the device to a system which delivers the implant to its location.

Preferably the device uses the stimulator anode and/or cathode as the power receiver features and incorporates a filter mechanism to prevent power from being directed at the stimulator generator circuit.

Preferably a device fixation and/or delivery feature directs electric field around or to the device.

Preferably the device includes an insulative coating to improve power transfer.

Preferably the device includes an insulative barrier between the hermetic housing and the fixation and/or delivery feature to improve power transfer.

Preferably one of more of the conductive features used to receive power and/or the device housing may form part of a battery housing

Preferably the conductive features are electrically coupled to the tissue by a capacitive or faradaic process.

Preferably the conductive features can be used to transfer data to the device by modulating the applied EMF.

Preferably the conductive features can be used to transfer data from the device to another device implanted or external to the body, by the device modulating the electrical potential in the tissue surrounding the device.

Preferably the conductive features comprise a treated surface. Preferably the treated surface is provided via a surface treatment process. The treated surface may increase the power transferred.

Preferably the conductive features are enhanced via surface treatment to direct the EMF to a particular or selected location on the device. Preferably this may improve performance or prevent interference with other device functions.

Preferably the treated surface may prevent corrosion of the conductive features.

Preferably the device incorporates a step-up converter or transformer to boost the received voltage. The boosted voltage may be a voltage required to operate the device.

In another aspect the disclosure provides an implantable medical device having an electrically conductive housing or casing part which comprises an electrode of a wireless power receiver wherein the electrode receives power by conducting current from surrounding tissue.

Preferably the implant is configured to be implanted by catheter or configured for catheterized delivery. The implant may be dimensioned or have an aspect ratio suitable for delivery via a vessel such as a blood vessel.

Preferably a further housing part comprises a further electrode of the wireless power receiver.

One or more conductive housing parts may be partially covered with an electrical insulator to provide a selected region of exposed conductive material for receiving current.

In some embodiments conductive regions or parts are provided at opposite ends of the implantable device.

The further housing part and/or electrode may comprise a geometric or location feature of the case or housing.

The location feature may comprise an anchoring mechanism or anchoring device.

Alternatively, a conductive element is provided dependent from the device, the conductive element comprising a further electrode of the wireless power receiver.

The conductive element may have a high aspect ratio (length, width or diameter). In some embodiments the conductive element comprises a wire.

The implantable device may comprise a pacemaker.

The housing part or electrode may comprise one or more conductive features.

Preferably one of more of the conductive features used to receive power and/or the device housing may form part of a battery housing

Preferably the conductive features are electrically coupled to the tissue by a capacitive or faradaic process.

Preferably an AC waveform is used to apply the EMF to the tissue for power transfer.

Preferably the conductive features can be used to transfer data to the device by modulating the applied EMF.

Preferably the conductive features can be used to transfer data from the device to another device implanted or external to the body, by the device modulating the electrical potential in the tissue surrounding the device.

Preferably the conductive features comprise a treated surface. Preferably the treated surface is provided via a surface treatment process. The treated surface may increase the power transferred.

Preferably the conductive features are enhanced via surface treatment to direct the EMF to a particular or selected location on the device. Preferably this may to improve performance or prevent interference with other device functions.

Preferably the conductive features are enhanced via surface treatment may to prevent corrosion of the conductive features.

Preferably the device incorporates a step-up converter or transformer to boost the received voltage. The boosted voltage may be a voltage required to operate the device.

Preferably one of more of the conductive features may additionally comprise a neurostimulator.

In another aspect the disclosure provides a wireless power transfer receiver having first and second electrodes configured to receive current from body tissue to provide power to a load.

Preferably the output impedance of the receiver is of the same order of magnitude as the impedance of the load. Preferably the impedances are substantially matched.

Preferably the load comprises a battery.

Preferably the load further comprises an implantable medical device.

Preferably the implantable device is implantable via a vessel in the body.

Preferably the implantable device comprises external conductive features.

Preferably the device is configured to receive power via the conductive features from an electromotive force applied to body tissue.

Preferably the conductive features comprise one or more exterior conductive surfaces.

Preferably the conductive features are separated by an insulator

Preferably one or more of the conductive features may comprise a locating feature of the device.

Preferably one or more of the conductive features may comprise an anchoring feature of the device.

Preferably one of more of the conductive features may perform a conductive function in addition to receiving power.

Preferably one of more of the conductive features may additionally comprise a pacemaker electrode.

Preferably the device receives power using a device fixation feature which anchors the device to a vessel or organ wall.

Preferably the device receives power using a delivery feature as one of the electrodes, the delivery feature being used to temporarily attach the device to a system which delivers the implant to its location.

Preferably the device uses the stimulator anode and/or cathode as the power receiver features and incorporates a filter mechanism to prevent power from be directed at the stimulator generator circuit.

Preferably a device fixation and/or delivery feature directs electric field around or to the device.

Preferably the device includes an insulative coating to improve power transfer.

Preferably the device includes an insulative barrier between the hermetic housing and the fixation and/or delivery feature to improve power transfer.

Preferably one of more of the conductive features used to receive power and/or the device housing may form part of a battery housing

Preferably the conductive features are electrically coupled to the tissue by a capacitive or faradaic process.

Preferably an AC waveform is used to apply the EMF to the tissue for power transfer.

Preferably the conductive features can be used to transfer data to the device by modulating the applied EMF.

Preferably the conductive features can be used to transfer data from the device to another device implanted or external to the body, by the device modulating the electrical potential in the tissue surrounding the device.

Preferably the conductive features comprise a treated surface. Preferably the treated surface is provided via a surface treatment process. The treated surface may increase the power transferred.

Preferably the conductive features are enhanced via surface treatment to direct the EMF to a particular or selected location on the device. Preferably this may to improve performance or prevent interference with other device functions.

Preferably the treated surface may prevent corrosion of the conductive features.

Preferably the device incorporates a step-up converter or transformer to boost the received voltage. The boosted voltage may be a voltage required to operate the device.

Preferably one of more of the conductive features may additionally comprise a neurostimulator.

In another aspect the disclosure provides a wireless power transfer system comprising:

a transmitter means configured to provide an electric field to body tissue

a receiver means having first and second electrodes configured to receive current through the body tissue.

In another aspect the disclosure provides a wireless power system primary apparatus comprising a first electrode and a second electrode in opposed relationship to the first electrodes, the electrodes being configured to apply an electromotive force to body tissue interposed between the electrodes.

Preferably the electrodes comprise plate-like structures.

Alternatively, each electrode comprises an array.

Preferably the electrodes are insulated.

Preferably the electrodes are configured as a wearable item.

In another aspect the disclosed subject matter provides a method of wireless power transfer comprising receiving power at a receiver device implanted in body tissue from current conducted through the body tissue.

Preferably the device is configured to receive power via the conductive features from an electromotive force applied to body tissue.

Preferably the conductive features comprise one or more exterior conductive surfaces.

Preferably the conductive features are separated by an insulator

Preferably one or more of the conductive features may comprise a locating feature of the device.

Preferably one or more of the conductive features may comprise an anchoring feature of the device.

Preferably one of more of the conductive features may perform a conductive function in addition to receiving power.

Preferably one of more of the conductive features may additionally comprise a pacemaker electrode.

Preferably the device receives power using a device fixation feature which anchors the device to a vessel or organ wall.

Preferably the device receives power using a delivery feature as one of the electrodes, the delivery feature being used to temporarily attach the device to a system which delivers the implant to its location.

Preferably the device uses the stimulator anode and/or cathode as the power receiver features and incorporates a filter mechanism to prevent power from be directed at the stimulator generator circuit.

Preferably a device fixation and/or delivery feature directs electric field around or to the device.

Preferably the device includes an insulative coating to improve power transfer.

Preferably the device includes an insulative barrier between the hermetic housing and the fixation and/or delivery feature to improve power transfer.

Preferably the method includes applying an electromotive force to body tissue to produce the current.

Preferably the electromotive force is generated using transmitter electrodes. Preferably the transmitter electrodes are provided adjacent to an external surface of the body tissue, such as skin.

Preferably one of more of the conductive features used to receive power and/or the device housing may form part of a battery housing.

Preferably the conductive features are electrically coupled to the tissue by a capacitive or faradaic process.

Preferably an AC waveform is used to apply the EMF to the tissue for power transfer.

Preferably the conductive features can be used to transfer data to the device by modulating the applied EMF.

Preferably the conductive features can be used to transfer data from the device to another device implanted or external to the body, by the device modulating the electrical potential in the tissue surrounding the device.

Preferably the conductive features comprise a treated surface. Preferably the treated surface is provided via a surface treatment process. The treated surface may increase the power transferred.

Preferably the conductive features are enhanced via surface treatment to direct the EMF to a particular or selected location on the device. Preferably this may improve performance or prevent interference with other device functions.

Preferably the treated surface may prevent corrosion of the conductive features.

Preferably the device incorporates a step-up converter or transformer to boost the received voltage. The boosted voltage may be a voltage to that required to operate the device.

Preferably one of more of the conductive features may additionally comprise a neurostimulator.

In another aspect the disclosed subject matter provides a method of wireless power transfer comprising applying an electromotive force to body tissue and receiving power at a receiver device implanted in the body tissue from current conducted through the body tissue.

Preferably the method comprises charging a battery in the implanted device.

In another aspect the disclosed subject matter includes a leadless cardiac pacemaker having a configured to power from current through the body tissue.

Preferably the power receiver circuit comprises a conditioning circuit. Preferably the conditioning circuit can include a boost circuit.

Preferably the boost circuit can be one or more of a transformer, DC-DC converter, multiplier.

Preferably the transformer includes a magnetic core.

Preferably the magnetic core is a toroid.

Preferably the transformer is designed for high frequency.

Preferably the boost circuit may include any existing commercially available ICs such as STM SPV1050.

Preferably the boost circuit may include under voltage and over voltage loop up.

Preferably the boost circuit can help reducing the device size.

Preferably the conditioner may include a maximum power point tracker.

Preferably the maximum power point tracker can include a load tracking mechanism.

Preferably the load can be any energy storage units such as batteries or super capacitors.

Preferably the fixation mechanism can be spiral.

Preferably the spiral mechanism can be similar or different to existing devices such as Nanostim™.

Preferably the spiral fixation is used to receive power.

Preferably the spiral fixation is a conductor.

Preferably the spiral fixation is electrically connected to the encapsulated device.

Preferably the spiral is electrical exposed.

Preferably the spiral is electrical insulated from any nearby fixations.

As used herein the term “and/or” means “and” or “or”, or both. As used herein “(s)” following a noun means the plural and/or singular forms of the noun. The term “comprising” as used in this specification means “consisting at least in part of”. When interpreting statements in this specification which include that term, the features, prefaced by that term in each statement, all need to be present, but other features can also be present. Related terms such as “comprise” and “comprised” are to be interpreted in the same manner. It is intended that reference to a range of numbers disclosed herein (for example, 1 to 10) also incorporates reference to all rational numbers within that range (for example, 1, 1.1, 2, 3, 3.9, 4, 5, 6, 6.5, 7, 8, 9 and 10) and also any range of rational numbers within that range (for example, 2 to 8, 1.5 to 5.5 and 3.1 to 4.7). The entire disclosures of all applications, patents and publications, cited above and below, if any, are hereby incorporated by reference.

The disclosed subject matter also provides method or system which may broadly be said to consist in the parts, elements and features referred to or indicated in this specification, individually or collectively, in any or all combinations of two or more of those parts, elements or features. Where specific integers are mentioned in this specification which have known equivalents in the art to which the invention relates, such known equivalents are deemed to be incorporated in the specification.

Other aspects of the invention may become apparent from the following description which is given by way of example only and with reference to the accompanying drawings.

### BRIEF DESCRIPTION OF THE DRAWINGS

FIG. 1 is a diagram showing a new conductive transcutaneous energy transfer system.

FIG. 2 is a diagram of the system if FIG. 1 in use as applied to a human torso.

FIG. 3 is a schematic diagram showing components of a transmitter apparatus.

FIG. 4 is a schematic diagram showing components of a receiver apparatus.

FIGS. 5, 5a, 6, 6a, and 6b are isometric and diagrammatic views of IMDs incorporating a power receiver system.

FIG. 7 is a general equivalent model schematic of cTET consisting of transmitter, receiver and tissue medium with the exclusion of any additional external circuitry.

FIG. 8 is a general equivalent model schematic lumping R13,23,14,24 into one component. The resulting component is a fictitious resistor that links the TX and RX side (coupling resistance). The coupling resistance can easily be calculated analytically as in equation 2.

FIG. 9 is a resistive equivalent circuit that includes a resistive coupling component.

FIG. 10a is a simplified impedance equivalent circuit of cTET.

FIG. 10b is a simplified admittance equivalent circuit of cTET.

FIG. 11 is an admittance two-port network for cTET.

FIG. 12 is a plot of maximum power transfer as a function of variation in inductance. The values are designed for 6.78 MHz operating frequency, 10 mm radius RXEs separated by 15 mm and 50 mm radius TXEs separated by 70 mm.

FIG. 13a is a Norton network of CCTET.

FIG. 13b is Thevenin network of CCTET.

FIG. 14 shows the geometry setup in COMSOL Multiphysics environment. The dimensions match the ones in practice. Any sweep in the geometry is done on this model.

FIG. 15 is a plot of power vs load variation for a fully compensated TX and RX at 27.5 mm separation. Analytical data is taken from equations (8-11, 14).

FIGS. 16A and 16B show a diagrammatic illustration of a receiver apparatus in plan view and isometric view respectively.

FIG. 16C shows a diagram of a switch network for use with the apparatus of FIGS. 16A and 16B

### DETAILED DESCRIPTION

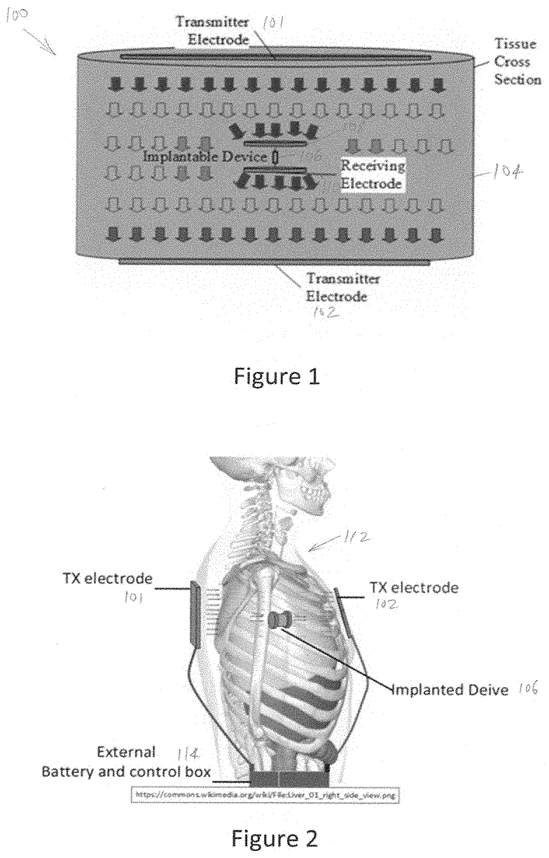

The subject matter disclosed herein provides a wireless power transfer system that employs a form of conductively coupled power transfer to transfer energy to deeply implanted devices (DIBDs). The system 100 is shown diagrammatically in FIG. 1 reveals a new circuit configuration wherein the power is applied using a transmitter system comprising two transmitter electrodes 101 and 102 which in at least some embodiments are disposed opposite to each other across tissue bulk 104. This configuration of transmitter electrodes differs from prior art configurations in which the transmitter electrodes are placed adjacent i.e. alongside each other in the same plane. The implantable device 106 is located deep within the tissue 104 and has receiving electrodes 108 and 100 which are electrically connected to the device 106.

Whilst power transfer to DIBDs is challenging due to low coupling, the system disclosed herein allows simplification of the power transfer model much like IPT. Based on the low coupling simplification and with the knowledge that tissue behaves largely as a conductor, a new model of power transfer is provided further below, namely Conductive Power Transfer (cTET). This model is used to design example systems which deliver useful amounts of power deep into the body, showing that cTET can be used to power deeply implanted devices.

The cTET system is depicted in FIG. 2. It consists of a pair of transmitter electrodes (TXE) 101 and 102 which is some embodiments are configured for placement across the human or animal body 102 (comprising tissues 104) near the implant location which in this example is within the patient's torso in the region of the chest. Thus, the electrodes 101 and 102 are placed on the back and front of the chest to power a device deep in the patient's torso. The transmitter electrodes 101 and 102 are electrically connected to an external unit 114 which includes a power supply together with appropriate control circuitry and may also include communication apparatus for communicating with the implanted device 106.

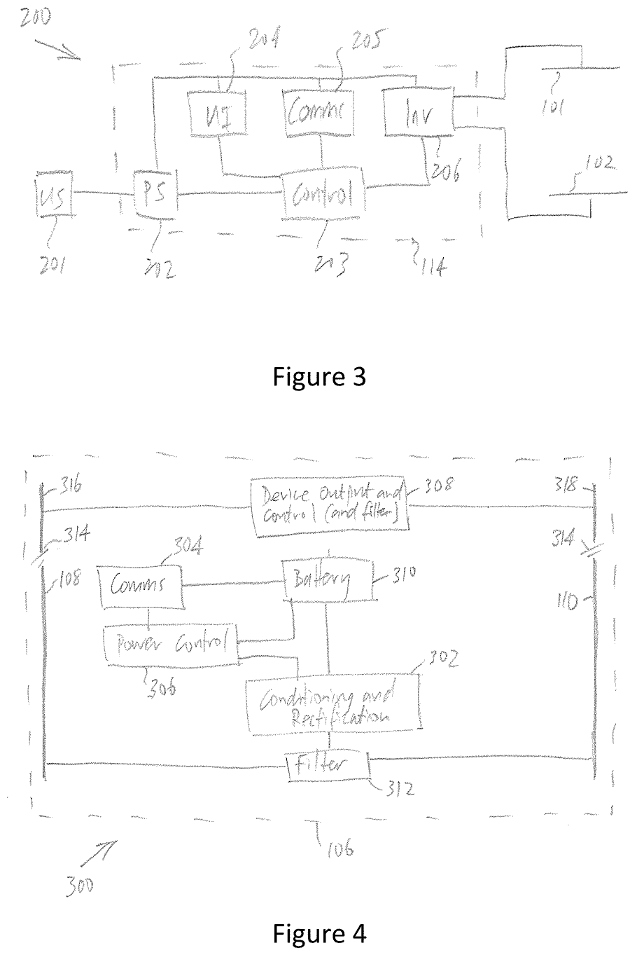

FIG. 3 is a diagram showing an example of components of the transmitter side 200 that may be used in some embodiments. A utility supply 201 can optionally be connected to the apparatus to provide power to a power supply 202 which may comprise a battery. Suitable control circuitry 203 which can comprise a microprocessor is provided for overall control of the transmitter apparatus. A User Interface 204 can be provided, along with a communication module or circuit 205 for communication with the receiver apparatus 300 as describer further below. cTET offers conductive communication over the power link from the implantable device to the outside world. Communication can be provided for any type of data transfer including power regulation. An inverter 206 has an output which is electrically connected to the transmitter electrodes 101 and 102, and is operable to provide an alternating electric potential between the electrodes 101 and 102 to thereby a time varying electric field (E-field) applied on the transmitting electrodes.

FIG. 4 is a diagram showing an example of the receiver apparatus 300 that may be used in some embodiments. In this example, the electrodes 108 and 110 may comprise external conductive features of an implantable device 106. External conductive features of the device 106 may also include functional conductors 316 and 318 which interact with the surrounding tissue or another device to provide a diagnostic and therapeutic function. In the example of a pacemaker the electrodes 316 and 318 may provide an electric signal for cardiac pacing. In other examples electrodes 316 and 318 may be configured to receive diagnostic information. The conductors 316 and 318 may be configured for a leadless pacemaker or other device.

As will be described further below, in some examples or embodiments the functional conductors 316 and 318 may also be used to receive power, or in other words, the power receiving electrodes 108 and 110 may comprise the same conductive feature or surface as the device functional electrodes 316 and 318. The line breaks 314 are present to indicate that in some embodiments the conductors (108 and 316) and (110 and 318) may be electrically connected and in others they may be separate conductors. In some embodiments conductors 108 and 316 may be electrically connected while conductors 110 and 318 are electrically separate, and in other embodiments conductors 110 and 318 may be electrically connected while conductors 108 and 316 are electrically separate.

The E-field produced by the transmitter electrodes generates a current flowing to the implanted receiver electrodes 108 and 110 via the surrounding partially conductive tissue 104. In embodiments in which the functional electrodes 316 and 318 also comprise the power receiving electrodes 108 and 110, a filter 312 may optionally be provided to ensure that there is no crossover between the signals provided or received by the device control and output circuit 308 and the power receiving circuitry. A filter may additionally or alternately be included in circuit 308.

As an example, the filter can prevent power from the power transfer system getting into the pacing system and so forth. So as to prevent power flowing through the pacing system reducing the risk of damage and corrosion to the pacing system. Furthermore, the filter can prevent pacing signals getting into the power receiver ports as this may load the pacer and reduce it functionally.

The power receiving electrodes 108 and 110 are electrically connected to either the filter 312 or a power receiving and conditioning circuit 302. In some embodiments circuit 302 may comprise a rectifier. In some embodiments circuit 302 may additionally or alternatively comprise a compensation or impedance matching network, and in some embodiments, it may comprise a filter. A primary function of the conditioning circuit 302 is to charge an energy storage device such as battery 310 and thus provide a power supply for operation of the device 106. The power supply derived from the battery and/or the conditioning circuit 302 can supply power to other modules or circuitry in the apparatus, such as the communication module 304, power control circuit 306 and the medical device control and functional output circuitry 308 which provides the required diagnostic or therapeutic function.

The communication circuit 304 may communicate with the transmitter apparatus 200 via the communication circuit 205. The communication may occur through a separate communications channel or use the electrodes 108 and 110 as a communications channel. Information that is communicated may include device control or status information such as power demand, or power availability, or battery status for example, but may also include patient diagnostic information monitored or collected by the device. The power control circuit 306 or device control circuit 308 for example may include a memory in which data is stored, and the stored data may be transmitted to the transmitter 200 as required.

In some embodiments, where power is provided to the electrodes or fixation/delivery parts, there is a capability of switching the connection of the receiver electrodes in order to align the receivers with the field, such that they can always receive power. This provides for electrical switching if the right positions of electrodes cannot be obtained.

Also, another useful feature for any implementation is the ability to use a filter to prevent power from the power transfer system getting into the pacing system and so forth. It would for instance be good to prevent power flowing through the pacing system which could damage it or cause corrosion. It would also be good to prevent pacing signals getting into the power receiver ports as this may load the pacer and reduce it functionally. For example, it would be good to have good coverage of the idea of having circuits or features (which might be a coating) that create isolation between the stimulator and power receiving parts.

In some embodiments the transmitting electrodes 101 and 102 can be fabricated metallic electrodes that are insulated, for example using an insulator such as Parylene, to avoid direct skin contact. In some embodiments the electrodes can be applied directly to skin i.e. non-insulating electrodes may be used. In other embodiments a conductive gel may be used.

Conveniently, in some embodiments existing electrodes may be used as the transmitter electrodes 101 and 102. For example, if a patient is wearing electrodes for other purposes (such as cardiac monitoring) then those electrodes may additionally be used as transmitter electrodes 101 and 102. Thus, the transmitter electrodes 101 and 102 may be adhesively mounted to a patient using a conductive or nonconductive adhesive, so that they remain in place despite patient movement.

The transmitter electrodes 101 and 102 can in some embodiments each comprise a plurality of electrodes. For example, one or both of the electrodes 101 or 102 may comprise multiple conductive members which may or may not be insulated but which may comprise an array and thus be distributed across a skin surface on one side of a tissue volume, so that the E field is distributed appropriately or efficiently across the tissue volume in which the E field is to be generated. The electrodes 101 and 102, may comprise a wearable item, for example being provided in a vest or harness.

As discussed above, the receiver apparatus 300 functions by the receiver electrodes 108 and 110 receiving an electric current via the partially conductive tissue 104. Therefore, means or mechanisms other than the electrodes 101 and 102 may be used to create the E field that generates the current. For example, the E field may be created using a dipole antenna or inductive coil as the transmitter apparatus. Electrical coupling at either of both of the transmitter electrodes and receiver electrodes may occur by capacitive or faradaic processes.

In some embodiments the receiving electrodes 108 and 110 can be placed on or in the immediate vicinity of the implant, and/or may comprise a part of the housing, or shell, or anchoring, or placement features, or geometry of the implanted device, as will be described further below.

As will be apparent from FIGS. 1 and 2, the use of electrodes that are located on either side of a tissue volume allows an E field to be created across the tissue volume bounded by the transmitter electrodes. Therefore, the receiver device may be located at any one of multiple different regions or locations within the tissue volume and receive power. Furthermore, multiple receiving devices may receive power from a single transmitter apparatus. cTET system disclosed herein generates a field which is predominantly uniform. Any receiver devices placed in the path will be capable of receiving energy. This is also discussed in connection with the loose coupling model disclosed further below. The advantage is that multiple devices, separate from each other, can be powered simultaneously. Powering a network of devices means one transmitter to many devices.

Many implanted devices are provided or packaged ready for implantation in a hermetically sealed housing or receptacle. This is usually titanium, which is a conductor and thus provides a material which is suitable for at least one of the receiver electrodes 108 or 110. Thus in some embodiments the housing of implanted device may provide one or both of the receiver electrodes 108 and 110.

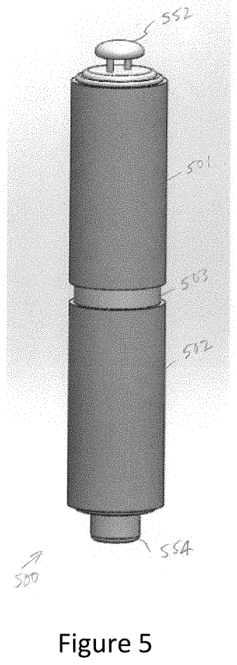

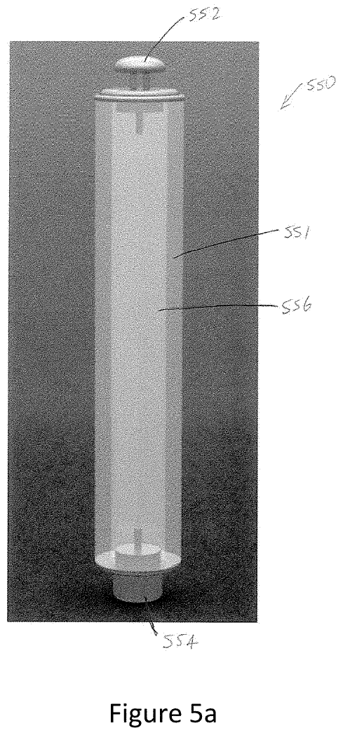

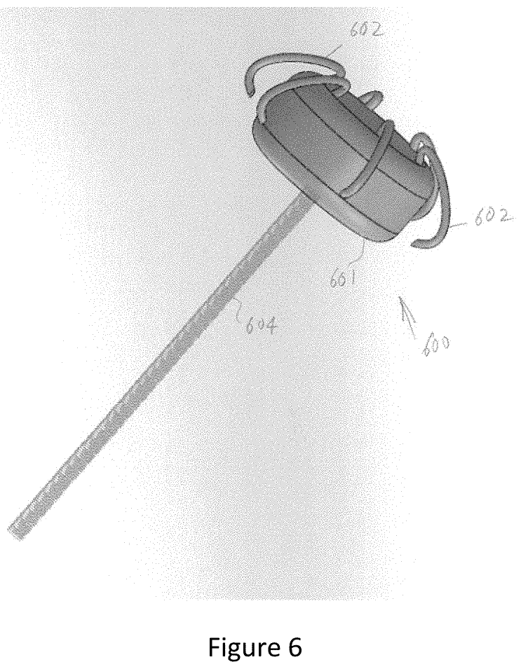

In some embodiments an implantable device including a receiver system 300 as described above or elsewhere in this document may be configured to be implantable by catherization, for example via a blood vessel. Examples are disclosed below with reference to FIGS. 5, 5a and 6.

FIG. 5 shows an implantable active device 500 which has a case or housing comprising a first electrically conductive part 501, a second electrically conductive part 502 and an electrical insulator part 503 which separates parts 501 and 502. Functional electrodes 552 and 554 provide or receive electrical signals for the diagnostic or therapeutic functions that the implanted device is intended to achieve. These correspond to the electrodes 316 and 318 described with reference to FIG. 4. Functional electrodes 552 and 554 may also comprise part of the housing.

Together the housing components 501, 502 and 503 provide a hermetically sealed case or housing without altering the overall dimensions of an existing device. The parts 501 and 502 correspond to and function as receiver electrodes 108 and 110. Therefore, the cTET system disclosed herein can be integrated to existing packaging technologies of implantable devices—typically titanium based cans—without the need for additional power receivers. This is in contrast to alternative technologies like IPT, in which additional coils are required (either inside the case or outside the case, or in a separate case). The advantages are that the size of the targeted devices will not be increased, and the complexity is not increased by having additional elements outside the hermetic can encapsulating the device.

In some embodiments it is desirable, although not absolutely necessary, to orientate the receiver apparatus so that electrode 108 of the receiver is nearest to electrode 101 of the transmitter and electrode 110 of the receiver is nearest to electrode 102 of the transmitter, or vice versa. This allows a shorter path for current to travel through the tissue 104 so there is likely to be more efficient power transfer. Also, increasing the distance between the electrodes 108 and 110, particularly if the electrodes are oriented so that receiver electrodes are closer to corresponding transmitter electrodes. A metallic implantable device can be made to receive power with improved performance by applying an insulation coating to the device to extend the effective distance between electrodes beyond what is available from the implant's native features, such as feedthroughs associated with stimulation or recording.

As another embodiment based on the FIG. 5 example, an insulative coating may be applied over housing parts 501 and 502 in regions close to the insulator 503, so that the exposed conductive surfaces of parts 501 and 502 are spaced further apart.

The use of an insulation or insulative coating on various parts of the case or housing advantageously makes the technology compatible with existing packages. That is, it allows reasonable voltages to be received, or it increases the power availability. This is a significant advantage as it means that an existing design can be translated to be conductively powered according to the system disclosed herein without adding new/costly packaging such as ceramic cylinders. Also, to keep the device small, in some embodiments the battery can is an exterior of part of the housing, meaning that some parts of the device cannot be made of ceramic without out size increase or new constructions for the battery.

FIG. 5a shows another example of an implantable device 550 which has a case 551 or housing comprising an insulator, for example a ceramic cylinder which forms an internal sealed volume 556 within which the components necessary to provide the required functionality are housed. In this embodiment, the functional electrodes 552 and 554 (which correspond to the electrodes 316 and 318 described in FIG. 4) are also used as the power receiving electrodes (i.e. electrodes 108 and 110 as described in FIG. 4). This has the benefit that the existing implantable device package can be used to perform a power receiving function without modification of the device formfactor. The ceramic or equivalent insulator can also be made shorter (for instance a conventional ceramic feedthrough) and an insulator coating applied to the conductive parts of the device in order to achieve separation of the power reception electrodes. For instance, anodization, ceramic or polymer coatings could be used to create a device which is made of a conductor, but which realizes separation of the power electrodes.

Surface treatment could also be applied to the conductive parts to enhance power transfer. The impedance of the electrode to tissue interface is a key factor in power transfer and can be enhanced by a number of processes such as surface roughening or deposition of a material with an improved impedance such as titanium nitride, gold and their equivalents. The surface treatment could additionally be used to reduce corrosion or enhance biocompatibility.

The construction of the receiver can affect impedance. Impedance matching for efficient power transfer is discussed further below. Matching can be affected using electrical components but can also be affected or at least adjusted through the surface finish of implantable devices. Thus, in some embodiments, impedance seen by the implantable device can be varied by insulating the surface in selected areas, which can improve power transfer capacity through the use of insulation layers without increasing device size or complexity.

Power transfer under cTET favours a large length to diameter ratio for the receiver device. This is a preferable arrangement to increase the voltage difference across the implantable device, as discussed above, and improve power transfer capacity. Devices can be very thin (even down to the thickness of a wire) and compatible with being delivered into the body using standard catherization delivery techniques. Adding a flexible piece of wire can be sufficient to enable an existing device to participate in effective power transfer. The wire can also be thought of as acting as a near field antennae. When devices require more power, or where continuous power is required, the use of an additional conductor can enhance power transmission. This also means device electronics can be made extremely small.

In some embodiments other features of existing casings or housings, such as locating features or anchoring devices or mechanisms, can be used to increase power transfer. In FIGS. 5 and 5a the delivery features may be incorporated into the physical design of electrodes 552 and 554 for example. One or more delivery features may be used to temporarily attach the device to a system that delivers the implant to its location. As mentioned above, the device may use one or more of the electrodes 552 and 554 as power receiver features and incorporate a filter mechanism to prevent power from be directed at the stimulator generator circuit.

The device fixation and/or delivery features can assist with directing electric field around or to the device. Thus, for example in some embodiments an implantable device 600 as shown in FIG. 6 has original casing 601 and anchoring apparatus comprising resilient conductive fingers 602. Casing 601 and fingers 602 can be used as one receiving electrode. The fingers 602 add a significant amount of additional electrically conductive area, extension or reach which will assist power transfer. Furthermore, an elongate conductive feature such as a wire 604 can be added (suitably insulated from the casing 601) as the other electrode. The length of the wire greatly contributes to the voltage difference that may be achieved in use which further improves power transfer. The wire is easily added and is suitable for standard catherization delivery techniques, such as delivery through a blood vessel for example.

Further disclosure of the use of existing IMD features for power reception is discussed below with reference to the examples shown in FIGS. 6a and 6b. Referring to those figures, examples of cardiac pacemakers are shown. These are similar to the devices described in FIGS. 5, 5a and 6, and like features have like reference numerals across the figures. In these examples the delivery/retrieval (552) and attachment/fixation (554) mechanisms are shown in more detail. The delivery and/or retrieval feature 552 is adapted for engagement with a delivery or retrieval device such as a snare and/or docking member which is advanceable from the lumen of a catheter. The catheter may be routed through a vessel such as the femoral vein via a femoral access site into the right atrium and thence into the right ventricle for example.

The attachment or location or fixation features 554 and 602 have the purpose of engaging the IMD with surrounding tissue. For example, feature 602 has fingers, hooks or tines which are configured to entangle or engage with trabeculae within the chamber of the heart. Feature 554 uses a screw type fixation mechanism shown in more detail in close-up view referenced 558. These fixation mechanisms are conductive and electrically exposed and can also be used to shape the electromagnetic field. However, the mechanism is best not to be in electrical contact with the other parts of the device. The fixation mechanism should be connected to the circuitry inside the encapsulation as described above.

The retrieval/delivery mechanism together with the fixation mechanism form a differential voltage as described above, while also performing other conductive roles, for example as neurostimulators, for communication purposes or other therapeutic or diagnostic purposes. Therefore, the present invention allows extended use of existing product components. This has advantages in terms of additional componentry not being required so the product dimensions can be kept as small as possible.

In some circumstances the voltage received at the power receiving electrodes of the implanted device may not be sufficient to charge or operate the device, so a voltage step-up conditioning circuit or system 560 may be needed in some embodiments. This circuit may comprise part of the circuitry 302 described above. System 560 may incorporate one or more of transformers, DC-DC converters, multipliers or any other circuits. Also, further circuits, such as existing chips like STM SPV1050, may be used to harvest energy. If a converter is used, it is advantageous to implement under voltage look up because the source impedance is relatively high.

To make the most use of the available power, in some examples a maximum power point tracker 562 can be used. This can for example be another DC-DC converter or any control system. It is highly suitable for a dynamic load like batteries or similar energy storage units. Depending on the application it will be seen by those skilled in the art that other types of control systems can be used, such as constant current, constant voltage, max efficiency etc.

Models have been created in COMSOL simulation environment. The models invoke field and circuit simulations. The electrical characteristic of each component is taken from approved and reviewed sources.

The NANOSTIM leadless pacemaker by Abbot was first of a kind of commercial leadless pacemaker. The product is on hold due to battery malfunction. A model of this device in COMSOL, demonstrated that cTET can used to transfer an appreciable amount of power (25 mW at a tissue depth of more than 100 mm) transcutaneously. It should be noted that leadless pacing consumes a maximum of 100 uW of power when running at highest capacity.

The MICRA pacemaker has been the most promising leadless pacemaker designed and developed by Medtronic. The device is smaller in dimension than NANOSTIM. It has successfully been deployed and can be found in the market. Currently the on-board battery lasts between 5 to 12 years. The battery life however is expected to be reduced with added functionality such as dual pacing. COMSOL was used to demonstrate the use of cTET for MICRA.
One of the key advantages of cTET is the ability to boost the power transfer using a thin piece of wire, to generate a greater voltage potential across the load. Or one may wish to think of this as a near field antenna. The simulation revealed that a 0.25 mm radius wire of 20 mm height (or length) helps to boost the power delivered to MICRA from 3 mW to 9 mW. The helps to demonstrate receiver devices that are long relative to their width or diameter can be used advantageously with cTET. This has advantages for cardiac application, in particular for devices intended for location in the left ventricle, since appropriately sized devices which have a high aspect ratio (ration of length to width) will not interfere with the normal functionality of the heart.

cTET Model

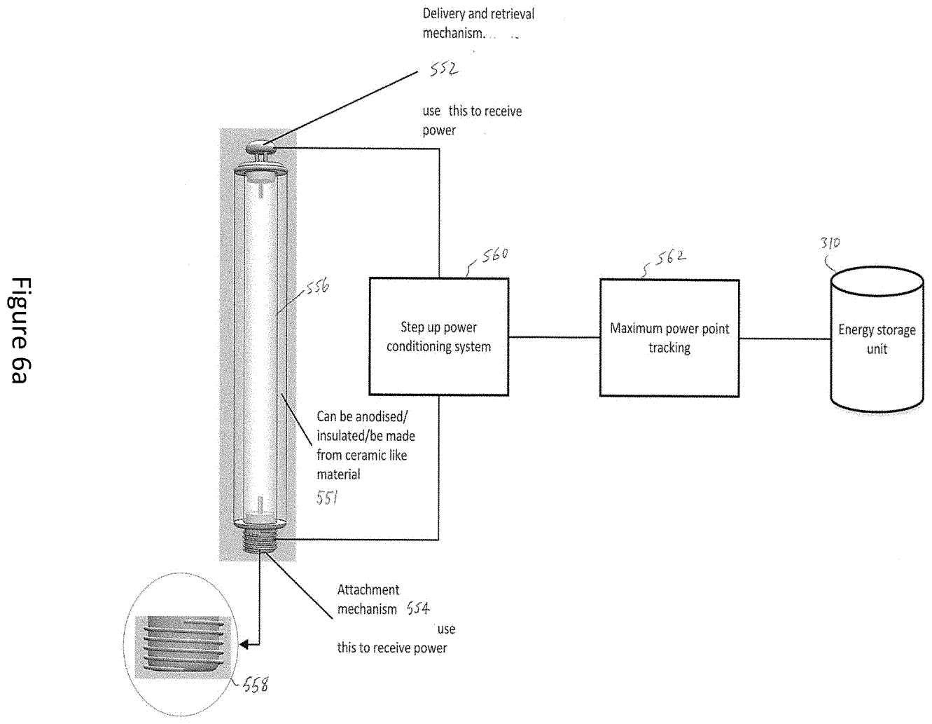

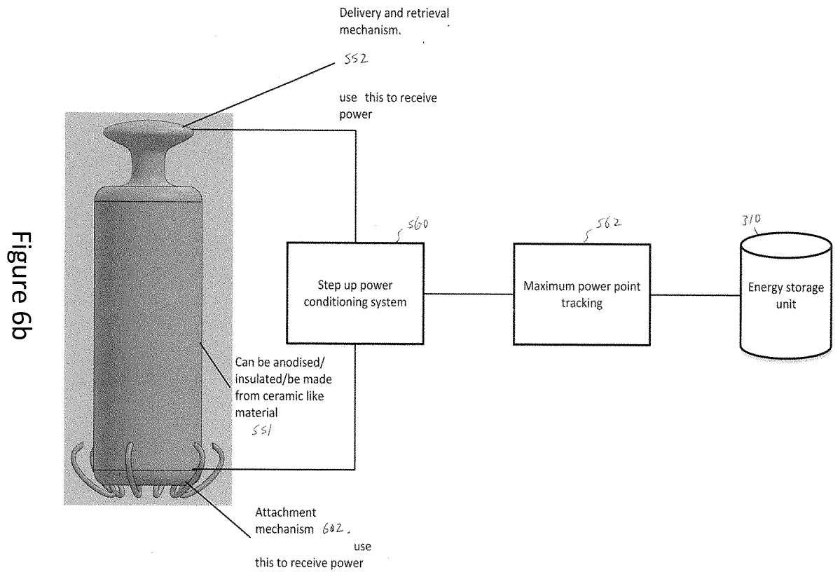

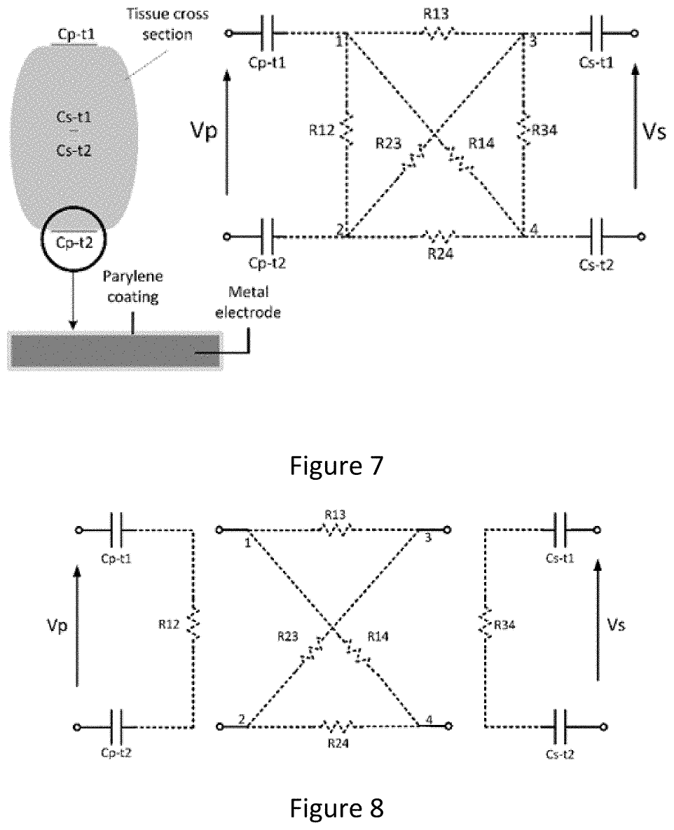

A model for the cTET system has been derived, and will now be discussed, beginning with reference to FIG. 7. One of the assumptions being made in this analysis is that tissue is significantly conductive (more than 90%). The capacitance formed between the transmitting electrodes (TXE, 101 and 102) and receiving electrodes (RXE, 108 and 110), and their respective insulation layers are represented as Cp-t # and Cs-t #.

The circuit model in FIG. 7 can be analysed as three main components, the transmitter, the medium (tissue 104) and the receiver. The transmitter side consists of TXEs which form Cp-t1-2 and R12, the medium includes R13,23,14,24 and finally the RXEs of the receiver side form Cs-t1-2 and R34.

To further simplify the equivalent model illustrated in FIG. 7, the transmitter (TX) and receivers (RX) sides can each be separately analysed, measured, linked via a coupling term. This involves measuring the impedance looking into the TXEs when RXEs are removed and vice versa.

To find the coupling term to link the transmitter and receiver sides, R13,23,14,24 are grouped into one component, namely Rc (illustrated in FIG. 8) which stands for coupling resistance. This term describes how much of the supplied power will be received on the implant side. Rc is dependent on the medium's electrical properties, dimensions of the TXEs and RXEs, and separation between the TXEs and RXEs.

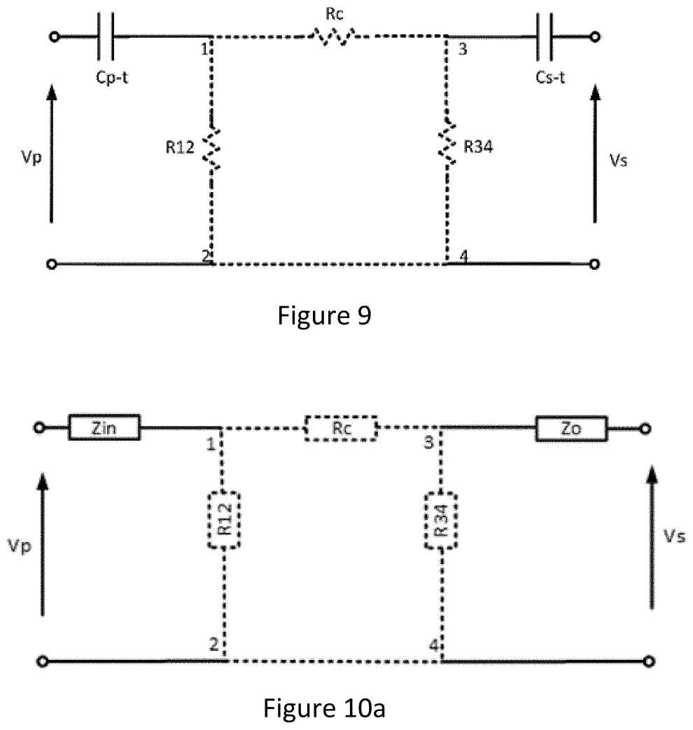

The electrode to tissue capacitances can also be lumped into Cp-t and Cs-t, the values of which can be analytically calculated in accordance with equation (1).

$$c = \frac{A . {ε}_{0} . {ε}_{r}}{d}$$

Where A is the surface area of electrodes, ε0 and εr are the free space and relative permittivity respectively and d is the thickness of the insulation layer. The final simplified circuit model is depicted in FIG. 9.

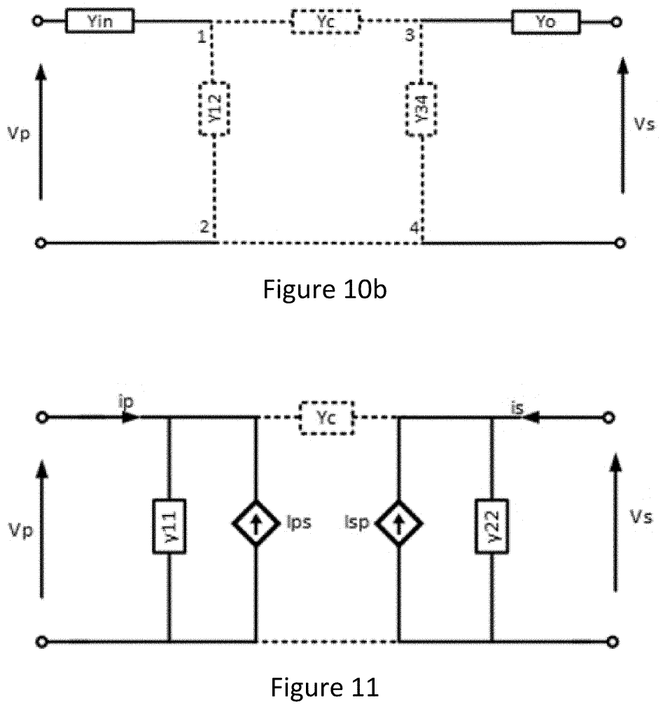

In a practical system, there are other factors such as lead inductance and parasitic components that have been neglected from the equivalent circuit in FIG. 9. Also, any type of impedance matching network can indisputably be added to the TXE and RXE terminals. To account for those, we will convert the electrode to tissue capacitance elements to impedance (Z) values, as illustrated in FIG. 10a. Each Z element can be a combination of reactive and resistive parts. It is noteworthy that the illustrated circuit schematic can alternatively be presented by its admittance equivalent as shown in FIG. 10b.

To calculate Z_{c} based on the equivalent circuit shown in FIG. 10a, we will apply nodal analysis. The equation (2) of R_{c} requires physical measurement of Vs when open-circuited (V_{soc}). This can either be done experimentally or via Multiphysics simulation. Finding V_{soc} is a common practice in WPT design, and so it has intentionally been deployed here. Equivalently, the admittance coupling Y_{c} can be derived using equation (3).

$${R}_{c} = \frac{{R}_{34} * ( \frac{{V}_{p} * {R}_{12}}{{z}_{in} + {R}_{12}} - {V}_{s ( o c )} )}{{V}_{s ( o c )}}$$

$${Y}_{c} = \frac{{V}_{s} ( o c ) * {Y}_{34}}{\frac{{V}_{p} * {Y}_{in}}{{Y}_{in} + {Y}_{12} - {V}_{s} ( oc )}}$$

Once R_{c} or Y_{c} are calculated, all the circuit elements are known, enabling the remaining parameters to be calculated. However, the circuit can be further developed into a two-port network to simplify the circuit design, as follows.

Admittance model of the two-port network FIG. 11 presents a current fed two-port network. In this network, the transmitting and receiving voltage-controlled current sources are defined as Ips=Vₛ·y₂₁ and Isp=Vₚ·y₁₂ respectively. y₁₁ and y₂₂ are the transmitting and receiving admittances while y₁₂ and y₂₁ are the reflected admittances from receiving side to the transmitting side and vice versa, respectively. (note the lower case).

$${y}_{11} = \frac{{Y}_{in} ( {Y}_{12} + \frac{{Y}_{c} ( {Y}_{34} + {Y}_{o} )}{{Y}_{c} + {Y}_{34} + {Y}_{o}} )}{{Y}_{in} + {Y}_{12} + \frac{{Y}_{c} ( {Y}_{34} + {Y}_{o} )}{{Y}_{c} + {Y}_{34} + {Y}_{o}}}$$

$${y}_{12} = - \frac{{Y}_{o} . {Y}_{c} . {Y}_{in}}{( {Y}_{34} + \frac{{Y}_{c} ( {Y}_{in} + {Y}_{12} )}{{Y}_{in} + {Y}_{c} + {Y}_{12}} + {Y}_{o} ) ( {Y}_{in} + {Y}_{c} + {Y}_{12} )}$$

$${y}_{21} = - \frac{{Y}_{in} . {Y}_{c} . {Y}_{o}}{( {Y}_{in} + {Y}_{12} + \frac{{Y}_{c} ( {Y}_{34} + {Y}_{o} )}{{Y}_{c} + {Y}_{34} + {Y}_{o}} ) ( {Y}_{c} + {Y}_{34} + {Y}_{o} )}$$

$${y}_{22} = \frac{{Y}_{o} ( {Y}_{34} + \frac{{Y}_{c} ( {Y}_{in} + {Y}_{12} )}{{Y}_{in} + {Y}_{12} + {Y}_{c}} )}{{Y}_{o} + {Y}_{34} + \frac{( {Y}_{in} + {Y}_{12} ) {Y}_{c}}{{Y}_{in} + {Y}_{12} + {Y}_{c}}}$$

Each of the parameters shown above is the function of frequency and geometrical size of the system. The geometry of the TXEs and RXEs, as well as their separation distance, will impact the amount of safe power that can be transferred. This will be discussed further below. The two-port network of FIG. 11, together with its simple to use governing equations, can be used to design cTETs of various shapes and size. The special case of loosely couple cTET will be discussed in the following subsections.

Compensation/Matching of cTET

In WPT systems, impedance matching is often used to achieve a certain goal. It can be to adjust systems sensitivity (quality factor), generate constant power on the load and or simply compensate for the reactive components. In cTET any of the mentioned targets can be achieved by designing additional circuitry. The easiest and the most obvious case is when the electrode to tissue capacitance of both sides are ideally compensated. If an ideal inductor on either side is assumed, we will have Yᵢₙ=∞ and Yₒ=∞. This condition will dramatically simplify the admittance parameters equations as follows:

y₁₁=Y₁₂+Y_{c} (8)

y₁₂=−Y_{c} (9)

Y₂₁=−Y_{c} (10)

y₂₂=Y₃₄+Y_{c} (11)

Not only the parametric equations look a lot simpler but also, they convey a clear message that the interaction between the transmitting side and the receiving side relies heavily on the coupling admittance Y_{c}. Therefore, to deliver more power, the coupling has to improve. To comply with the loosely coupling condition, however, Y_{c} shall remain less than Y₁₂. This will be revisited in the next sub-section.

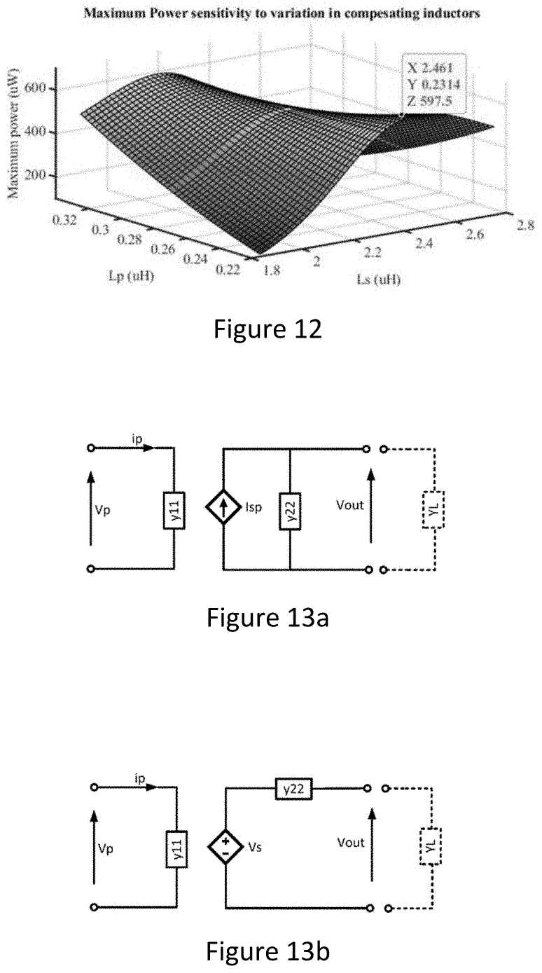

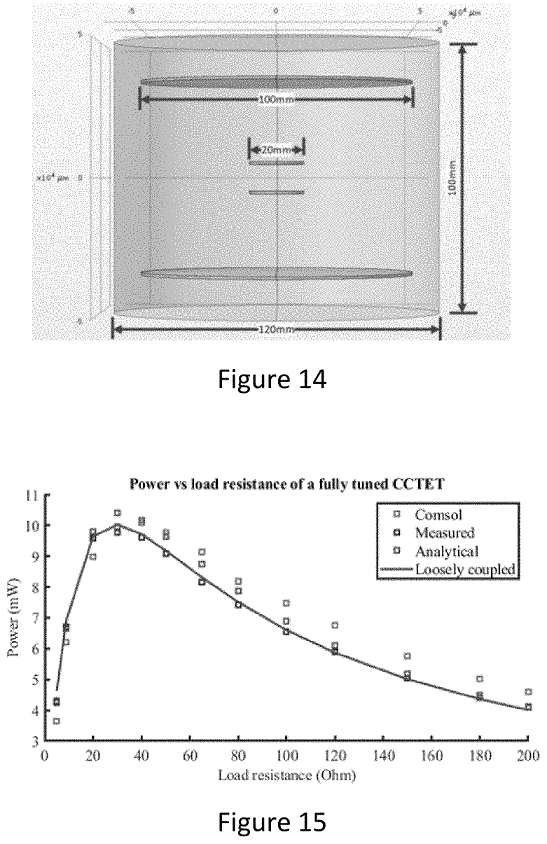

To understand how the TX and RX inductors collectively affect the maximum power transfer, the following surface plot in FIG. 12 is generated for a system with the dimensions similar to FIG. 14 with the respective RXEs and TXEs separation of 15 mm and 70 mm. The figure shows the maximum power transferred to a load vs the variation of the tuning inductors in 20% of its original optimum value (i.e the inductance required to make resonance). The load was varied to always achieve MPTP, in accordance with equation (15). The figure highlights the importance of each inductor; illustrating that the system is more sensitive to variations of the receiver tuning circuit. The plot also shows a promising 600 uW by only applying 2.5 V peak sinewave at 6.78 MHz on the TXEs.

A Loosely Coupled System

In the special case of loose coupling, the interaction between TX and RX circuits will weaken meaning the power drawn by the implant is insignificant. Under these conditions, the effect of the RX on TX will be negligible which will allow for each side (RX and TX) to be designed independently of the other. Referring to the two-port network in FIG. 11, the TX and RX sides become independent as Y_{c} tends to zero (no coupling).

For example, if y₁₁>>Y_{c} (Z_{c}>>Z₁₁), when shorting and opening the RX terminals, the impedance change seen at the TXEs will be unnoticeable. In other words, when Y₁₂/Y_{c}>>1 (where Y₁₂ is taken from FIG. 10b, and is the impedance across TXE) the cTET system can be thought of as loosely coupled. Therefore, in the case of DIBDs loose coupling conditions are met due to the large separation and small coupling to the implant.

For a loosely coupled system, it is appropriate to replace the coupling admittance with a current-controlled current source as shown in FIG. 13a. This can be converted to a Thevenin equivalent as shown in FIG. 13b.

In FIG. 13a:

Iₛₚ=k·Vₚ·y₂₂ (12)

Or in the Thevenin equivalent of FIG. 13b:

Vₛ=k·Vₚ (13)

In the above equation, k is the coupling factor and it is defined as the ratio of the input voltage to the output voltage.

Achieving Maximum Power Transfer in a Loosely Coupled cTET

Maximum power transfer (MPTP) for a loosely coupled system is satisfied when the load impedance matches the RX output impedance (y₂₂=YL or R₂₂=RL). This can be achieved by compensating for any reactance and matching the load resistance to the remaining real component of the impedance. Therefore, the respective MPTP conditions for the Thevenin and Norton models are:

$${P}_{max} = {[ \frac{{V}_{s ( oc ) .} {R}_{L}}{{R}_{22} + {R}_{L}} ]}^{2} * \frac{1}{{R}_{L}} = \frac{{V}_{s ( oc )}^{2}}{4. {R}_{L}} = \frac{{( k . {V}_{p} )}^{2}}{4. {R}_{L}}$$

$${P}_{max} = {[ \frac{\vert {I}_{s p} \vert . {Y}_{L}}{{y}_{22} + {Y}_{L}} ]}^{2} * \frac{1}{{Y}_{L}} = \frac{{\vert {I}_{s p} \vert}^{2}}{4 {Y}_{L}}$$

The equations in the last two subsections show that a loosely coupled cTET can be characterized by measuring the TX impedance, the RX impedance and measuring the open circuit voltage. The TX and RX can then be tuned individually to compensate for the reactance at the desired operating frequency. The load resistance can then be set to match the real component of the RX impedance to achieve maximum power transfer.

From the foregoing it can be seen that cTET is modelled like an impedance divider. Applying the maximum power transfer principle, the impedance of the load can be matched to the impedance seen by the implanted device. Reactive matching can also be used to ensure that power transfer remains conductive both on the transmitting and receiving ends.

This has significant benefits in enhancing power transfer. This is valuable as it delivers maximum power for a given geometry, or it can be seen to deliver maximum power for a given drive current applied externally. This means that the implanted device achieves maximum available power whilst remaining safe. Significantly, it has been shown that the impedance of the pickup is effectively resistive with a small contribution from the capacitance and/or lead inductance. Compensation is achieved by applying the complex conjugate load impedance. e.g. by adding a reactance plus making the implant load match the pickup resistance, such as by using a transformer, a dc/dc converter or a matching network. Therefore, selecting a load impedance which is of the same order of magnitude as the receiver output impedance facilitates efficient power transfer.

FIG. 14 shows a model that was used as the basis to validate the foregoing analysis.

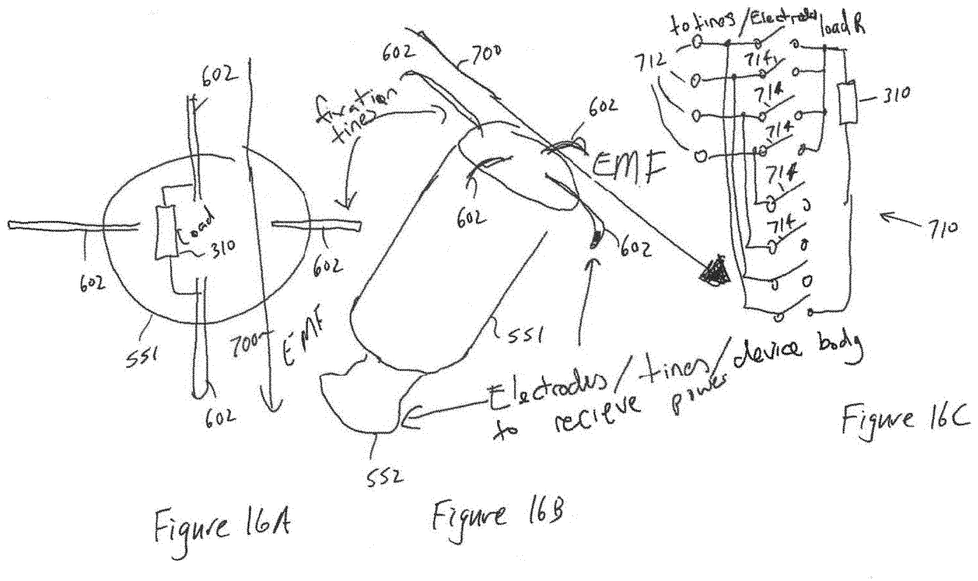

In some embodiments or examples the conductive regions or features may be selectively coupled with the load supplied by the device dependent on the power requirement or depending on the most efficient alignment of the conductive regions with the applied field. For example, a device like that shown in FIG. 6B is illustrated in FIG. 16A (in plan from above) and in FIG. 16B (in isometric view) along with an arrow 700 which represents the direction of the EMF or predominant field direction experienced by the device. The load shown diagrammatically by way of example in FIG. 16A is shown as comprising battery 310 (which will be inside the device) but may include other components supplied with energy from the electric field or conduction region surrounding the device. FIG. 16C shows a switch array 710 having terminals 712 for connection to the conductive features (in this example tines 602) and switches 714 which are configured or operable to connect the conductive features individually or completely or in selected combinations, with the load or loads. In this way the conductive features which are best aligned with the field or applied EMF or are in best conductive contact with surrounding tissues may be selected for connection with the load or loads in order to maximise, or adjust, power received by the device.

A Fully Compensated Safe cTET Implementation

A fully compensated cTET system working in the safe operating region is now presented. The electrode to tissue capacitance of TX is compensated with an addition of a 1.6 nF COG capacitor and 428 nH inductor. The capacitance of the receiving side (RX) is compensated with a 2.3 uH inductance wound around a toroid core (Fair-Rite 5967000601). Full compensation condition has been confirmed by measuring resonant frequency with an impedance analyser. The CS1070 power amplifier is limited to 1A output current and therefore the voltage output has been lowered as the result to avoid clipping.

FIG. 15 shows the plot of the variation of load for 10 mm RXE and 50 mm TXE radii fixed at 27.5 mm (TXE-RXE) separation. The plot includes numerical simulation, measured, analytical and loosely coupled calculation results. The voltage input is set to 6.4 Vpk-pk sinewave operating at 6.7 MHz. The resulting RMS E-field is 32V/m which is 12% of the maximum possible exposure outlined in IEEE C95.1. The results show 10 mW of power delivered with an efficiency of 0.4% (TXE to RXE). The CS1070 power amplifier has 4 W of dissipated power which has been neglected here given that in practice a high-efficiency inverter would be used. The majority of the energy to generate a voltage on the RXEs is dissipated in the saline tank which corresponds to SAR and tissue heating. Despite low efficiency, the system is compatible with safety standards as the losses are spread throughout the tissue relatively evenly giving low SAR except near the implant.

The conductive nature of the tissue 104 allows for a simple, easy to use and insightful analytical model. The presented model simplifies the contribution of tissue to resistance only as capacitance is insignificant in the low MHz range. It also introduces the concept of resistive coupling, which is easily modelled and measured, yet gives accurate predictions of power transfer. This leads to a two-port representation of cTET which can be used to predict power form either simulation or measured impedances.

For deeply implanted devices, the two-port model was simplified to account for loose coupling which allows separation of the TX and RX equivalent circuits. This further simplifies the analysis to a resistance divider model which remains accurate when compared to measurements and simulations. The model also allows for a compensation circuit to be designed and maximum power to the load to be calculated.

Simulation of SAR shows that useful amounts of power can be delivered deep into the body. For instance, power is available is nearly constant as RX separation increases. This is a significant result because it makes cTET a feasible option to power deeply implanted devices. SAR peaks limit power transfer being concentrated near the TX or RX electrodes. This is a result of the conductive nature of the tissue which works as a resistor divider. For large depths of power transfer, the driving voltage must be increased, however this does not lead to higher peak SAR as the voltage is absorbed by the tissue bulk.

The present disclosure shows how the cTET model can be simplified down to an impedance divider for useful product design. In addition, a model has been created that allows the decomposition of the transmitter and receiver. cTET can charge implantable medical devices such as pacemakers while they are operational.
interruption to the device therapy delivery e.g. the pacemaker can continue to provide pacing.

Because the tissue is largely resistive in nature, power transfer is nearly independent of frequency. Other wireless power delivery methods use highly reactive power transfer methods which means that they are tuned to resonance to cancel the reactance and improve the power transfer. This is very difficult as there are two separate tuned systems which can differ over time or with location or proximity to one another. this technique has very little reactance, meaning the implant pickup is not reactive or resonant (unless it is desired and engineered in). this means that tuning of the external source (typically to counter lead inductance or electrode capacitance) can be done without concern for the implant frequency.

A capacitively coupled conductive wireless power transfer (cTET) method based on conductive tissue to power deeply implanted biomedical devices (DIBDs) is demonstrated. The cross-sectional geometry of cTET minimises skin losses that makes the technology suitable for deep implantation.

The various illustrative logical blocks, modules, routines, and algorithm steps described in connection with the embodiments disclosed herein can be implemented as electronic hardware (e.g., ASICs or FPGA devices), computer software that runs on computer hardware, or combinations of both. Moreover, the various illustrative logical blocks and modules described in connection with the embodiments disclosed herein can be implemented or performed by a machine, such as a processor device, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A processor device can be a microprocessor, but in the alternative, the processor device can be a controller, microcontroller, or state machine, combinations of the same, or the like. A processor device can include electrical circuitry configured to process computer-executable instructions. In another embodiment, a processor device includes an FPGA or other programmable device that performs logic operations without processing computer-executable instructions. A processor device can also be implemented as a combination of computing devices, e.g., a combination of a DSP and a microprocessor, a plurality of microprocessors, one or more microprocessors in conjunction with a DSP core, or any other such configuration. Although described herein primarily with respect to digital technology, a processor device may also include primarily analog components. For example, some or all of the rendering techniques described herein may be implemented in analog circuitry or mixed analog and digital circuitry. A computing environment can include any type of computer system, including, but not limited to, a computer system based on a microprocessor, a mainframe computer, a digital signal processor, a portable computing device, a device controller, or a computational engine within an appliance, to name a few. The elements of a method, process, routine, or algorithm described in connection with the embodiments disclosed herein can be embodied directly in hardware, in a software module executed by a processor device, or in a combination of the two. A software module can reside in RAM memory, flash memory, ROM memory, EPROM memory, EEPROM memory, registers, hard disk, a removable disk, a CD-ROM, or any other form of a non-transitory computer-readable storage medium. An exemplary storage medium can be coupled to the processor device such that the processor device can read information from, and write information to, the storage medium. In the alternative, the storage medium can be integral to the processor device. The processor device and the storage medium can reside in an ASIC. The ASIC can reside in a user terminal. In the alternative, the processor device and the storage medium can reside as discrete components in a user terminal.

Conditional language used herein, such as, among others, “can,” “could,” “might,” “may,” “e.g.,” and the like, unless specifically stated otherwise, or otherwise understood within the context as used, is generally intended to convey that certain embodiments include, while other embodiments do not include, certain features, elements or steps. Thus, such conditional language is not generally intended to imply that features, elements or steps are in any way required for one or more embodiments or that one or more embodiments necessarily include logic for deciding, with or without other input or prompting, whether these features, elements or steps are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list.

Disjunctive language such as the phrase “at least one of X, Y, or Z,” unless specifically stated otherwise, is otherwise understood with the context as used in general to present that an item, term, etc., may be either X, Y, or Z, or any combination thereof (e.g., X, Y, or Z). Thus, such disjunctive language is not generally intended to, and should not, imply that certain embodiments require at least one of X, at least one of Y, and at least one of Z to each be present.

While the above detailed description has shown, described, and pointed out novel features as applied to various embodiments, it can be understood that various omissions, substitutions, and changes in the form and details of the devices or algorithms illustrated can be made without departing from the spirit of the disclosure. As can be recognized, certain embodiments described herein can be embodied within a form that does not provide all of the features and benefits set forth herein, as some features can be used or practiced separately from others. The scope of certain embodiments disclosed herein is indicated by the appended claims rather than by the foregoing description. All changes which come within the meaning and range of equivalency of the claims are to be embraced within their scope.

Any routine descriptions, elements or blocks in the flow diagrams described herein and/or depicted in the attached figures should be understood as potentially representing modules, segments, or portions of code which include one or more executable instructions for implementing specific logical functions or elements in the routine. Alternate implementations are included within the scope of the embodiments described herein in which elements or functions may be deleted, or executed out of order from that shown or discussed, including substantially synchronously or in reverse order, depending on the functionality involved as would be understood by those skilled in the art.

It should be emphasized that many variations and modifications may be made to the above-described embodiments, the elements of which are to be understood as being among other acceptable examples. All such modifications and variations are intended to be included herein within the scope of this disclosure and protected by the following claims.

### Preferred Embodiments

1. An implantable device implantable via a vessel in the body, the implantable device comprising external conductive features and being configured to receive power via the conductive features from an electromotive force applied to body tissue.

2. An implantable device of claim 1 wherein an AC waveform is used to apply the EMF to the tissue of the body for power transfer.

3. An implantable device of claim 1 or 2 wherein the conductive features comprise one or more exterior conductive surfaces.

4. An implantable device of any one of claims 1 to 3 wherein the conductive features are separated by an insulator.

5. An implantable device of any one of claims 1 to 4 wherein one or more of the conductive features comprises a locating feature of the device.

6. An implantable device of any one of claims 1 to 5 wherein one or more of the conductive features comprises an anchoring feature of the device.

7. An implantable device of any one of claims 1 to 6 wherein one of more of the conductive features performs a conductive function in addition to receiving power.

8. An implantable device of any one of claims 1 to 7 wherein one of more of the conductive features additionally comprises a pacemaker electrode.

9. An implantable device of any one of claims 1 to 8 wherein one or more of the conductive features additionally comprise a neurostimulator.

10. An implantable device of any of one of claims 1 to 9 wherein the device receives power using a device fixation feature that anchors the device to a vessel or organ wall.

11. An implantable device of any of one of claims 1 to 10 wherein the device receives power using a delivery feature as one of the electrodes, the delivery feature being used to temporarily attach the device to a system which delivers the implant to its location.

12. An implantable device of any of one of claims 1 to 11 wherein the device uses a stimulator anode and/or cathode as a power receiver and incorporates a filter mechanism to prevent power from be directed at a stimulator generator circuit.

13. An implantable device of any one of claims 1 to 12 wherein the device fixation and/or delivery feature directs an electric field around or to the device.

14. An implantable device of any one of claims 1 to 13 wherein the device includes an insulative coating to improve power transfer.

15. An implantable device of any one of claims 1 to 14 wherein the device has a hermetic housing.

16. An implantable device of any one of claims 1 to 15 wherein the device includes an insulative barrier between the hermetic housing and the fixation and/or delivery feature to improve power transfer.

17. An implantable device of any one of claims 1 to 16 wherein one of more of the conductive features used to receive power and/or the device housing may form part of a battery housing.

18. An implantable device of any one of claims 1 to 17 wherein the conductive features are electrically coupled to the tissue by a capacitive process.

19. An implantable device of any one of claims 1 to 18 wherein the conductive features are electrically coupled to the tissue by a faradaic process.

20. An implantable device of any one of claims 1 to 19 wherein the conductive features can be used to transfer data to the device by modulating the applied EMF.

21. An implantable device of any one of claims 1 to 20 wherein the conductive features can be used to transfer data from the device to another device implanted or external to the body, by the device modulating the electrical potential in the tissue surrounding the device.

22. An implantable device of any one of claims 1 to 21 wherein the conductive features comprise a treated surface.

23. An implantable device of any one of claims 1 to 22 wherein the treated surface is provided via a surface treatment process.

24. An implantable device of claim 22 or 23 wherein the treated surface increases the power transferred by the device.

25. An implantable device of any one of claims 1 to 21 wherein the conductive features are enhanced via surface treatment to direct the EMF to a particular or selected location on the device.

26. An implantable device of claim 25 wherein the surface treatment improves performance or prevents interference with other device functions.

27. An implantable device of any one of claims 22 to 26 wherein the treated surface prevents corrosion of the conductive features.

28. An implantable device of any one of the claims 1 to 27 wherein the device incorporates a step-up converter or transformer to boost the received voltage.

29. An implantable device of claim 28 wherein the boosted voltage is the voltage required to operate the device.

### Further Preferred Embodiments

1. A wireless power transfer receiver having first and second electrodes configured to receive current from body tissue to provide power to a load.

2. A wireless power transfer receiver of claim 1 wherein the output impedance of the receiver is of the same order of magnitude as the impedance of the load.

3. A wireless power transfer receiver of claim 2 wherein the impedances are substantially matched.

4. A wireless power transfer receiver of any one of claims 1 to 3 wherein the load comprises a battery.

5. A wireless power transfer receiver of claims 1 to 4 wherein the load further comprises an implantable medical device.

6. A wireless power transfer receiver of claim 5 wherein the implantable device is implantable via a vessel in the body.

7. A wireless power transfer receiver of claim 5 or 6 wherein the implantable device comprises external conductive features.

8. A wireless power transfer receiver of claim 7 wherein the receiver is configured to receive power via the conductive features from an electromotive force applied to body tissue.

9. A wireless power transfer receiver of claim 7 or 8 wherein the conductive features comprise one or more exterior conductive surfaces.

10. A wireless power transfer receiver of any one of claims 7 to 9 wherein the conductive features are separated by an insulator.

11. A wireless power transfer receiver of any one of claims 7 to 10 wherein one or more of the conductive features comprises a locating feature of the receiver.

12. A wireless power transfer receiver of any one of claims 7 to 11 wherein one or more of the conductive features comprises an anchoring feature of the receiver.

13. A wireless power transfer receiver of any one of claims 7 to 12 wherein one of more of the conductive features performs a conductive function in addition to receiving power.

14. A wireless power transfer receiver of any one of claims 7 to 13 wherein one of more of the conductive features additionally comprises a pacemaker electrode.

15. A wireless power transfer receiver of any one of claims 12 to 14 wherein the receiver receives power using the anchoring feature that anchors the device to a vessel or organ wall.

16. A wireless power transfer receiver of any one of claims 1 to 15 wherein the receiver receives power using a delivery feature as one of the electrodes, the delivery feature being used to temporarily attach the receiver to a system that delivers the implant to its location.

17. A wireless power transfer receiver of any one of claims 1 to 16 wherein the receiver uses a stimulator anode and/or cathode as the power receiver features and incorporates a filter mechanism to prevent power from be directed at a stimulator generator circuit.

18. A wireless power transfer receiver of any one of claims 1 to 17 wherein a device fixation and/or delivery feature directs electric field around or to the device.

19. A wireless power transfer receiver of any one of claims 1 to 18 wherein the receiver includes an insulative coating to improve power transfer.

20. A wireless power transfer receiver of any one of claims 1 to 19 wherein the receiver is enclosed in a hermetic housing.

21. A wireless power transfer receiver of any one of claims 1 to 19 wherein the receiver includes an insulative barrier between the hermetic housing and the fixation and/or delivery feature to improve power transfer.

22. Preferably one of more of the conductive features used to receive power and/or the device housing to form part of a battery housing.

23. Preferably the conductive features are electrically coupled to the tissue by a capacitive or faradaic process.

24. Preferably an AC waveform is used to apply the EMF to the tissue for power transfer.

25. Preferably the conductive features can be used to transfer data to the device by modulating the applied EMF.

26. Preferably the conductive features can be used to transfer data from the device to another device implanted or external to the body, by the device modulating the electrical potential in the tissue surrounding the device.

27. Preferably the conductive features comprise a treated surface. Preferably the treated surface is provided via a surface treatment process. The treated surface may increase the power transferred.

28. Preferably the conductive features are enhanced via surface treatment to direct the EMF to a particular or selected location on the device.

29. Preferably this may to improve performance or prevent interference with other device functions.

30. Preferably the treated surface may prevent corrosion of the conductive features.

31. Preferably the device incorporates a step-up converter or transformer to boost the received voltage.

32. Preferably the boosted voltage may be a voltage required to operate the device.

33. Preferably one of more of the conductive features may additionally comprise a neurostimulator.

34. A wireless power transfer system comprising:

a transmitter means configured to provide an electric field to body tissue, and a receiver means having first and second electrodes configured to receive current through the body tissue.

35. A wireless power system primary apparatus comprising a first electrode and a second electrode in opposed relationship to the first electrodes, the electrodes being configured to apply an electromotive force to body tissue interposed between the electrodes.

36. Preferably the electrodes comprise plate-like structures.

37. Alternatively, each electrode comprises an array.

38. Preferably the electrodes are insulated.

39. Preferably the electrodes are configured as a wearable item.

40. A method of wireless power transfer comprising receiving power at a receiver device implanted in body tissue from current conducted through the body tissue.

41. Preferably the device is configured to receive power via the conductive features from an electromotive force applied to body tissue.

42. Preferably the conductive features comprise one or more exterior conductive surfaces.

43. Preferably the conductive features are separated by an insulator

44. Preferably one or more of the conductive features may comprise a locating feature of the device.

45. Preferably one or more of the conductive features may comprise an anchoring feature of the device.

46. Preferably one of more of the conductive features may perform a conductive function in addition to receiving power.

47. Preferably one of more of the conductive features may additionally comprise a pacemaker electrode.

48. Preferably the device receives power using a device fixation feature which anchors the device to a vessel or organ wall.

49. Preferably the device receives power using a delivery feature as one of the electrodes, the delivery feature being used to temporarily attach the device to a system which delivers the implant to its location.

50. Preferably the device uses the stimulator anode and/or cathode as the power receiver features and incorporates a filter mechanism to prevent power from be directed at the stimulator generator circuit.

51. Preferably a device fixation and/or delivery feature directs electric field around or to the device.

52. Preferably the device includes an insulative coating to improve power transfer.

53. Preferably the device includes an insulative barrier between the hermetic housing and the fixation and/or delivery feature to improve power transfer.

54. Preferably the method includes applying an electromotive force to body tissue to produce the current.

55. Preferably the electromotive force is generated using transmitter electrodes. Preferably the transmitter electrodes are provided adjacent to an external surface of the body tissue, such as skin.

56. Preferably one of more of the conductive features used to receive power and/or the device.

57. Preferably the housing may form part of a battery housing.

58. Preferably the conductive features are electrically coupled to the tissue by a capacitive or faradaic process.

59. Preferably an AC waveform is used to apply the EMF to the tissue for power transfer.

60. Preferably the conductive features can be used to transfer data to the device by modulating the applied EMF.

61. Preferably the conductive features can be used to transfer data from the device to another device implanted or external to the body, by the device modulating the electrical potential in the tissue surrounding the device.

62. Preferably the conductive features comprise a treated surface. Preferably the treated surface is provided via a surface treatment process. The treated surface may increase the power transferred.

63. Preferably the conductive features are enhanced via surface treatment to direct the EMF to a particular or selected location on the device. Preferably this may improve performance or prevent interference with other device functions.

64. Preferably the treated surface may prevent corrosion of the conductive features.

65. Preferably the device incorporates a step-up converter or transformer to boost the received voltage. The boosted voltage may be a voltage to that required to operate the device. Preferably one of more of the conductive features may additionally comprise a neurostimulator.

66. A method of wireless power transfer comprising applying an electromotive force to body tissue and receiving power at a receiver device implanted in the body tissue from current conducted through the body tissue.

67. Preferably the method comprises charging a battery in the implanted device.

68. In another aspect the disclosed subject matter includes a leadless cardiac pacemaker having a power receiver circuit configured to power from current through the body tissue.

69. Preferably the power receiver circuit comprises a conditioning circuit.

70. Preferably the conditioning circuit can include a boost circuit.

71. Preferably the boost circuit can be one or more of a transformer, DC-DC converter and multiplier.

72. Preferably the transformer includes a magnetic core.

73. Preferably the magnetic core is a toroid.

74. Preferably the transformer is designed for high frequency.

75. Preferably the boost circuit may include any existing commercially available ICs such as STM SPV1050.

76. Preferably the boost circuit may include under voltage and over voltage loop up.

77. Preferably the boost circuit can help reducing the device size.

78. Preferably the conditioner may include a maximum power point tracker.

79. Preferably the maximum power point tracker can include a load tracking mechanism.

80. Preferably the load can be any energy storage units such as batteries or super capacitors.

81. Preferably the fixation mechanism can be spiral.

82. Preferably the spiral mechanism can be similar or different to existing devices such as Nanostim™.

83. Preferably the spiral fixation is used to receive power.

84. Preferably the spiral fixation is a conductor.

85. Preferably the spiral fixation is electrically connected to the encapsulated device.

86. Preferably the spiral is electrically exposed.

87. Preferably the spiral is electrical insulated from any nearby fixations.

1. A medical device implant having an electrically conductive housing or casing part which comprises an electrode of a wireless power receiver wherein the electrode receives power by conducting current from surrounding tissue.

2. A medical device implant of claim 1 wherein the implant is configured to be implanted by catheter or configured for catheterized delivery or the implant is dimensioned for delivery via a vessel such as a blood vessel.

3. (canceled)

4. A medical device implant of claim 1 wherein a further housing part on the implant comprises a further electrode of the wireless power receiver.

5. A medical device implant of claim 1 wherein one or more conductive housing parts are at least partially covered with an electrical insulator to provide a selected region of exposed conductive material for receiving current.

6. A medical device implant of claim 1 wherein the conductive regions or parts are provided at opposite ends of the implantable device.

7. A medical device implant of claim 1 wherein the conductive regions or parts are provided at the same end of the implantable device, are separated from each other yet in the same plane.

8. A medical device implant of claim 4 wherein the further housing part and/or electrode comprise an anchor.

9. (canceled)

10. A medical device implant of claim 1 wherein a conductive element is provided dependent from the device, the conductive element comprising a further electrode of the wireless power receiver.

11. (canceled)

12. A medical device implant of claim 10 wherein the conductive element comprises a wire.

13. A medical device implant of claim 1 wherein the implantable device acts as a pacemaker.

14. A medical device implant of claim 1 wherein the housing part or electrode comprise one or more conductive features.

15. A medical device implant of claim 14 wherein one or more of the conductive features are electrically couplable to the surrounding tissue by a capacitive or faradaic process.

16. (canceled)

17. (canceled)

18. A medical device implant of claim 14 wherein the conductive features are configured to transfer data from the device by modulating the electrical potential in the tissue surrounding the device.

19. A medical device implant of claim 14 wherein the conductive features comprise a treated surface configured to increase the power transferred to the implant.

20. (canceled)

21. (canceled)

22. A medical device implant of claim 14 wherein the conductive features direct the received power to a particular or selected location on the device.

23. (canceled)

24. (canceled)

25. A medical device implant of claim 1 wherein the device comprises a step-up converter or transformer to boost the voltage of the received power.

26. (canceled)

27. A medical device implant of of claim 1 comprising an energy storage device, wherein the received power is configured to charge the energy storage device.

28. A wireless power transfer system comprising:
a transmitter means configured to provide an electric field to body tissue, and
a receiver means having first and second electrodes configured to receive current through the body tissue.

29. A wireless power system primary apparatus comprising a first electrode and a second electrode in opposed relationship to the first electrodes, the electrodes being configured to apply an electromotive force to body tissue interposed between the electrodes.

30. A wireless power transfer system of claim 28 wherein the receiver comprises an energy storage device, wherein the received current is configured to charge the energy storage device.

31. (canceled)
